(19) United States
(12) Patent Application Publication
(10) Pub. No.: US 2026/0367042 A1
(43) Pub. Date: Nov. 19, 2026

(54) ACOUSTIC FORCE ELASTOGRAPHY MICROSCOPY

(71) Applicant: Mayo Foundation for Medical Education and Research, Rochester, MN (US)

(72) Inventors: Hsiao-Chuan Liu, Rochester, MN (US); Matthew W. Urban, Rochester, MN (US)

(21) Appl. No.: 19/103,306

(22) PCT Filed: Aug. 14, 2023
(86) PCT No.: PCT/US2023/072174

§ 371 Date: Feb. 12, 2025

Related U.S. Application Data
(60) Provisional application No. 63/397,683, filed on Aug. 12, 2022.

(51) Int. Cl. G01N 29/06; G01N 29/24

(52) U.S. Cl. CPC G01N 29/0681; G01N 29/2418; G01N 2291/02827

(57) ABSTRACT

An acoustic radiation elastography microscope includes an ultrasound transducer that generates an acoustic radiation force (ARF) and a suitable motion detection system, such as an optical coherence tomography (OCT) system or an ultrasound system that measures particle motion of shear waves induced in a sample by the ARF.

### STATEMENT OF FEDERALLY SPONSORED RESEARCH

This invention was made with government support under DK092255 awarded by the National Institutes of Health. The government has certain rights in the invention.

### BACKGROUND

Tissue mechanics have been comprehensively investigated for the past two decades with elastography techniques because tissue mechanical properties are significantly associated with disease states. So far, two well-known modalities to evaluate mechanical properties of ultrathin biological samples (smaller than 1 mm) are atomic force microscopy (AFM) and acoustic radiation force optical coherence elastography (ARF-OCE). Although AFM is a powerful tool for the quantification of mechanical properties of soft tissues and cells, it includes the risk that samples could be regionally damaged by the compression from a scanning cantilever, has a high cost maintenance fee, and is time-consuming in measurement. ARF-OCE is another common modality used to evaluate mechanical properties of thinner tissues.

Previous works describing ARF-OCE have been published; however, these prior techniques have drawbacks. The range of mechanical properties of biological tissues that can be evaluated is largely decided by attenuation and OCT field of view. It is a global measurement. That being said, the mechanical properties measured from a small region can represent the properties of entire samples. It cannot represent each localized mechanical property. However, to evaluate mechanical properties with optical methods, optical scatterers are essentially needed. The ARF-OCE will be limited if the biological samples or specimen are optically transparent. A small sample (such as 5 mm) could make mechanical property measurements with wave propagations to be inaccurate due to boundary issues.

### SUMMARY OF THE DISCLOSURE

The present disclosure addresses the aforementioned drawbacks by providing an acoustic force elastography microscope system, which includes a translation stage configured to receive a material sample and to translate the material sample along a first spatial dimension and a second spatial dimension orthogonal to the first spatial dimension; an ultrasound transducer configured to generate an acoustic radiation force (ARF) directed at a surface of the material sample; and an optical coherence tomography system configured to measure particle motion attributable to shear wave motion in the material sample caused by the ARF. Additionally or alternatively, an ultrasound system may be used to measure particle motion attributable to shear wave motion in the material sample caused by the ARF.

It is another aspect of the present disclosure to provide a method for acoustic force elastography microscopy. The method includes generating an acoustic radiation force (ARF) with an ultrasound transducer. The ARF is exerted on a surface of a material, thereby generating surface waves that travel outwards on the surface and a longitudinally polarized shear wave that travels through a thickness of the material. Elastography data are acquired using an optical coherence tomography system to measure particle motion of reflected longitudinally polarized shear waves generated by the ARF. An elasticity map can be reconstructed from the elastography data.

The foregoing and other aspects and advantages of the present disclosure will appear from the following description. In the description, reference is made to the accompanying drawings that form a part hereof, and in which there is shown by way of illustration one or more embodiments. These embodiments do not necessarily represent the full scope of the invention, however, and reference is therefore made to the claims and herein for interpreting the scope of the invention.

### BRIEF DESCRIPTION OF THE DRAWINGS

FIG. 1A illustrates an example acoustic force elastography microscope (AFEM).

FIG. 1B illustrates an example AFEM system using an optical coherence tomography (OCT) system as the motion detection system. The three function generators are utilized to provide trigger and driving signals for synchronizing the AFEM scan (Function Generator 1), acoustic excitation (Function Generator 2), and recording signals with the OCT system (Function Generator 3). The details of red dashed rectangle are illustrated in FIG. 1A to explain the behaviors of longitudinally polarized shear waves (LPSWs) and reflected longitudinally polarized shear waves (RLPSWs). The LPSWs are generated by the acoustic radiation force and travel through a thin scaffold with thickness h. The RLPSWs are detected at the surface by OCT. GM: galvanometer mirror, RF: radiofrequency.

FIG. 1C illustrates an example AFEM scan pattern. The elastography resolution in the lateral and elevational direction on a scaffold with X by Y mm scan range represents Δk and Δs, respectively. p: an individual elastography measurement position, i: the number of M-scans, j: the number of B-scans, z: depth, k: total scan numbers of lateral elastography measurement positions, s: total scan numbers of elevational elastography measurement positions, Δk: elastography resolution in the lateral direction, Δs: elastography resolution in the elevational elastography direction, X: sample width, Y: sample length.

FIG. 2 shows the mechanism of wave velocity evaluation of acoustic force elastography microscope (AFEM).

FIG. 3 illustrates an example in which two thin materials were used to demonstrate the performance of AFEM.

FIG. 4 shows an example of the time of arrival of the peak shown in the profile.

FIG. 5 shows an example in which the data size for each measurement can be small using AFEM. These measurements use a lateral range of 2 mm.

FIG. 6 illustrates a demonstration of creating a two-dimensional (2D) elastography map using AFEM.

FIG. 7 illustrates an evaluation of the elastography resolution of AFEM in a gelatin hydrogel with high mechanical contrast.

FIG. 8 is a flowchart setting forth the steps of an example AFEM method according to some aspects of the present disclosure.

FIG. 9 is a block diagram of an example ultrasound system that may form a part of an AFEM system according to some aspects of the present disclosure.

### DETAILED DESCRIPTION

Described here are systems and methods for acoustic force elastography microscopy (“AFEM”). In general, an acoustic radiation force (ARF) is directed at a surface of a material sample, which generates mechanical waves (e.g., shear waves) that propagate in the sample. The particle motion attributable to the shear wave, or other mechanical wave, motion in the material sample caused by the ARF is then measured or otherwise detected using a suitable detection system. As one example, the detection system may be an optical coherence tomography system. As another example, the detection system may be an ultrasound system, which in some instances may be the same ultrasound system used to generate the ARF, or may be a different ultrasound system.

The systems and methods described in the present disclosure are advantageous for evaluating the mechanical properties of thin materials (e.g., hydrogel scaffolds, tissue-mimicking gelatin hydrogels, oligo[poly(ethylene glycol)fumarate] (OPF) hydrogels, OPF hydroxyapatite (HA) nanocomposites, and so on) over a two-dimensional (2D) region for tissue engineering and other applications. For example, the systems and methods described in the present disclosure can be used to evaluate mechanical properties of scaffolds corresponded with cell differentiation, cross-linking densities, mineralization, biodegradation, and osteocalcin expression as examples in tissue engineering. AFEM can be used to evaluate elastic properties of transparent samples; that is, the sample material does not need to include scatterers unlike other non-contact elastography measurement techniques. Additionally, AFEM provides a higher elastography spatial resolution as compared with dynamic mechanical analysis techniques. Furthermore, AFEM systems and methods described in the present disclosure do not require direct contact with the sample, which allows for longitudinal studies within the same sample.

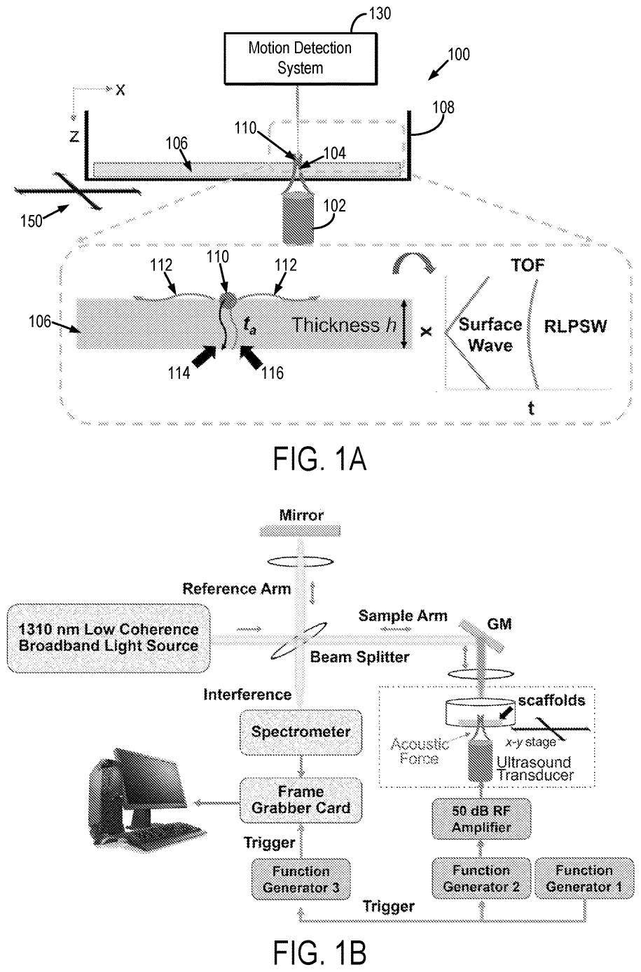

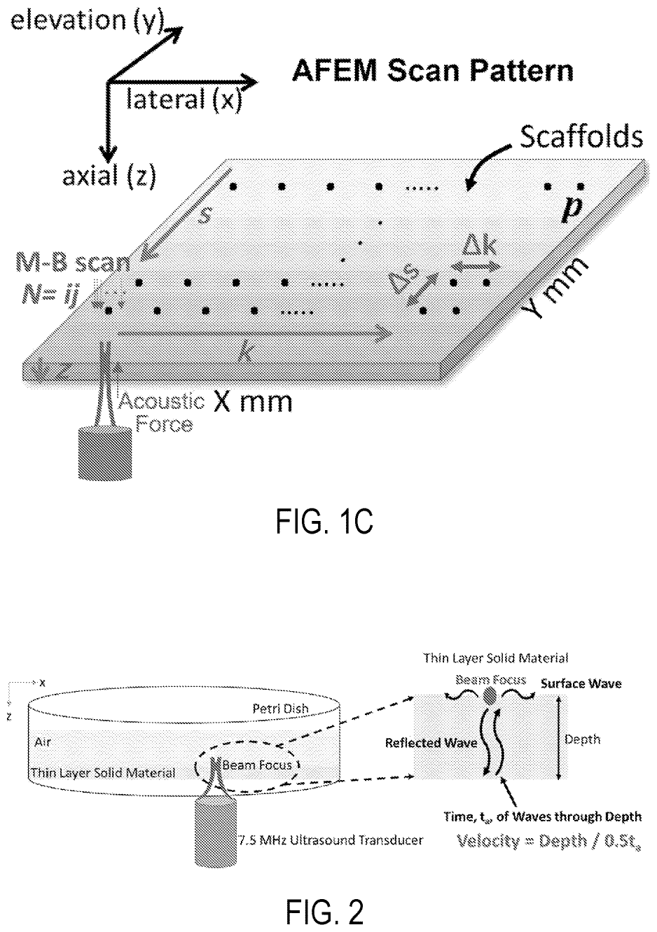

In general, an AFEM system 100 can include an ultrasound transducer 102 to generate acoustic force, a motion detection system 130 (e.g., an optical coherence tomography (OCT) system, an ultrasound system), and a translation stage 150 (e.g., an x-y translation stage). In the example AFEM system 100 illustrated in FIG. 1A, a focused ultrasound transducer 102 is used to produce an acoustic radiation force (ARF) 104 to generate mechanical waves in a material sample 106. The material sample 106 may include a thin transparent scaffold, as an example. The focused ultrasound transducer 102 may be coupled to a container 108 for holding the material sample 106. As one example, the sample container 108 may include a Petri dish, or the like. The focused ultrasound transducer 102 may be coupled to the sample container 108 using ultrasound coupling gel, or the like.

The ARF 104 is exerted on the surface of material sample 106 to generate mechanical waves in the material sample 106. For instance, the ARF 104 is directed to a focal point 110 to generate surface waves 112 that travel outwards from the focal point 110 on the surface of the material sample 106 as well as mechanical waves that travel through the thickness of the material sample 106, which may be defined as a longitudinally polarized shear waves 114 (LPSW). The mechanical waves that reflect from the bottom of the sample container 108 and propagate back to the surface of the material sample 106 are reflected LPSWs (RLPSWs) 116. The arrival time, tₐ, of the RLPSW is determined by the local thickness of the material sample 106, h, and shear modulus, μ, of the material according to the relationship:

$${t}_{a} = \frac{1}{2} \cdot h \cdot \sqrt{\frac{ρ}{μ}} ; \qquad ( 1 )$$

where ρ is the mass density of the material. In some examples, the mass density may be assumed as 1000 kg/m³ for biological samples. The factor of one-half in Eqn. (1) accounts for the round-trip travel distance. Following from Eqn. (1), the shear modulus of the material can be given by:

$$μ = {ρ ( \frac{h}{2 {t}_{a}} )}^{2} ; \qquad ( 2 )$$

where the term h/2tₐ may be defined as the velocity of RLPSWs, C_{AFEM}.

By assuming that the material is isotropic, homogeneous, linear, and nearly incompressible, Young's modulus, E, can be determined by E≈3μ as the localized thickness of the material, h, and arrival time, tₐ, of the RLPSWs are obtained. The spatial resolution of AFEM can be determined based on a lateral focal size of the ultrasound transducer 102, which as one example can be calculated by:

2.44×f×λ_{US} (3);
where f is the f-number defined as the focal distance divided by the aperture size of the transducer and λ_{US} is the ultrasound wavelength. Measurement of RLPSWs at the surface of materials do not require optical scatterers, i.e., materials can be transparent, which is common for hydrogel scaffolds in tissue engineering.

As described above, a motion detection system 130 is used to detect and otherwise measure the motion of mechanical waves (e.g., surface waves, LPSWs, and/or RLPSWs) generated in the material sample 106 by the ARF 104. The motion detection system may be an OCT system, an ultrasound system, or other suitable system for detecting the motion of mechanical waves in a thin material sample.

Referring now to FIG. 1B, an example AFEM system 100 in which the motion detection system 130 is an OCT system is illustrated. The role of the OCT in the AFEM system 100 is to measure particle motion of RLPSWs. OCT has a number of advantages including being a non-contact measurement with micro-scale spatial resolutions, sensitivity to sensing particle displacements in nanometer scales with flexible scan speeds; therefore, it is capable of supporting a finer elastography resolution in the AFEM system. FIG. 1B illustrates the basic optical layout of a spectral-domain optical coherence tomography (SD-OCT) system (e.g., TEL320C1, Thorlabs Inc., Newton, NJ, USA). The OCT system is equipped with a 1300 nm source with low coherence broadband (236.8 nm of bandwidth) and LK 4 lens kit (Thorlabs Inc., Newton, NJ, USA) to produce 20 μm of lateral resolution and 3.5 μm of z-axis resolution in air. A low coherence broadband source is split into a reference beam directed toward a stationary reference mirror and a sample beam directed toward the material sample 106. The back-reflected and back-scattered light from the material sample 106 and retroreflected light from the reference mirror are recombined by a coupler. A spectral interferogram is formed by spectrometers and data are collected by a frame grabber card. The fast Fourier transform (FFT) is utilized to form an A-scan from the receiver array in the SD-OCT system. Each pixel includes a real value and an imaginary value (in-phase/quadrature, IQ) from which magnitude and phase can be calculated. One-dimensional (1D) autocorrelation may be used to calculate the phase information, Δφ, of particle displacements including RLPSWs and surface waves. The particle velocity of RLPSWs V(z,t) are given by:

$$V ( z , t ) = \frac{Δ ϕ ( x , z , t ) {f}_{S R} {λ}_{O C T}}{4 π n} ; \qquad ( 4 )$$

where f_{SR} is the OCT scan rate, λ_{OCT} is center wavelength of the light source, and n is the refractive index of the material sample 106. Generally, the refractive index of biological materials, tissues, and tissue-mimicking phantoms ranges from 1.35 to 1.55.

The elastic properties of hydrogels used for tissue engineering are significantly associated with cell spreading and differentiation. Conventionally used compressional mechanical testing methods that are currently used to characterize mechanical properties of hydrogels have many limitations. The AFEM described in the present disclosure can be used to characterize the elastic mechanical properties of scaffolds and thin-layered engineered tissues for various applications in tissue engineering.

Scaffolds play an important role in tissue engineering, providing a supporting structure to mimic a native extracellular matrix (ECM) micro-environment for cell adhesion, proliferation, and migration. Mechanical properties of scaffolds are associated with cell differentiation, mineralization, and polymer molecular weights; therefore, characterizing scaffold mechanical properties are critical. Dynamic mechanical analysis (DMA) is the gold standard method to characterize hydrogel mechanical properties. However, the major limitations of DMA are destructive tests, contact with samples, and bulk elastic measurements, which leads to difficulties for making measurements in cell-laden hydrogels and conducting longitudinal studies to understand the elastic evolution of neat or cell-laden scaffolds. Although existing contactless elastography methods based on the shear wave elastography (SWE) technique to quantify mechanical properties of biological materials are widely used, scatterers are essential for measurement of the wave propagation. Scaffolds in tissue engineering can be transparent and relatively thin, so contactless elastography methods may be difficult to apply for mechanical characterization.

Using the AFEM systems and methods described in the present disclosure, the mechanical properties of neat, cell-laden, and growth factor-induced cell-laden scaffolds can be evaluated for tissue engineering applications. As a non-limiting example, AFEM employs highly focused ultrasound to produce acoustic forces exerted on scaffolds to generate longitudinally polarized shear waves (LPSWs) with a motion detection method, such as phase-sensitive optical coherence tomography (PS-OCT), to measure reflected LPSWs. The AFEM method is contactless with samples and non-destruction tests, which allows for longitudinal studies of the same samples. Moreover, AFEM does not require the samples to have optical scatterers for motion tracking and the mechanical properties can be evaluated over a two-dimensional (2D) region to provide a high elastography resolution for heterogeneous samples or cell-laden hydrogels, as examples.

The optical coherence tomography (OCT) system, or other motion detection system, and acoustic transducer can be accurately aligned. An x-y stage, or other translation stage, is used to manage the position of samples so that the scan range can be flexible and not limited by the OCT, or other motion detection system, field-of-view. The wave velocity traveling inside thin samples relies on the time of reflected waves coming from the boundary; therefore, no optical scatterers are needed and AFEM can be used to evaluate transparent samples. The mechanical properties of each localized position on samples can be individually determined to reflect a real distribution of Young's modulus, or other mechanical properties, in ultrathin tissues like microscope performance. AFEM achieves a high resolution elastography compared with traditional ARF-OCE. AFEM provides a robust tool in the applications of tissue engineering and regenerative medicine.

The following advantages can be achieved using AFEM. The AFEM system and data acquisition can provide high elastography resolution measurements with high throughput. Elastic properties of various structures and materials can be characterized, including tissue-mimicking phantoms of varying thickness and concentrations, neat scaffolds, and composite scaffolds. Elastic properties of cell-laden scaffolds and bone morphogenetic protein-2 (BMP-2) derived peptide-induced cell-laden scaffolds can also be characterized using AFEM, and their crosslinking with biomarkers can be explored over time for longitudinal studies. This versatile AFEM with numerous advantages is promising for the applications in tissue engineering, biomaterial and biology fields, including exploring mechanical properties of scaffolds with polymers, cell-encapsulated scaffolds, hydrogel stiffness quantification, mineralization, hydrogel degradation, as well as potentially exploring drug resistance regulated by matrix stiffness or mechanogenomics mechanisms to potentially explain the genome mutation rate with tissue stiffness for understanding cell infiltration as examples.

In an example configuration of an AFEM system, such as the AFEM system 100 shown in FIGS. 1A and 1B, an ultrasound transducer 102 for generating an acoustic radiation force exerted on the surface of the samples to generate wave propagation can include a 7.5 MHz highly focused single-element transducer transmitting 3750 cycles (500 μs). The focal distance of the transducer may be 11.84 mm measured by a pulse-echo test and an f-number of 1.07 can be obtained by the definition of the focal distance divided by an 11 mm aperture size of the transducer. A sinusoid burst signal may be generated by a function generator and may be amplified 50 dB by a radiofrequency (RF) power amplifier to drive the transducer. A 140 kHz A-scan rate with a motion detection system 130 implemented as an optical coherence tomography (OCT) system can be used to obtain a high image quality.

The three function generators illustrated in the example system configuration in FIG. 1B are utilized to provide trigger and driving signals for synchronizing the AFEM scan, acoustic excitation, and recording signals. For example, function generator 3 (33500 B, Keysight, SantaRosa, CA, USA) may control the OCT scan rates and triggering in the external mode. Function generator 1 (33250A, Agilent, Santa Clara, CA, USA) may be a master trigger to synchronize the timing for the entire AFEM system. Function generator 2 (33250 A, Agilent, Santa Clara, CA, USA) may provide the sinusoidal burst signal with two different pulse durations to provide signals that can be tracked with sufficient amplitude.

The acoustic radiation force is generated by a 7.5 MHz ultrasound transducer and the force is exerted on the surface of thin layer solid material to generate the surface waves and reflected waves. The reflected waves are measured by the motion detection system, which in the example shown in FIG. 1B is an OCT system. The OCT image acquired with the OCT system also records the depth information of the thin material.

For measuring tissue-mimicking gelatin hydrogels as the system test stage, a customized scan pattern in AFEM was designed to use a 50 kHz A-line OCT scan rate and 100 μm lateral spacing over a 16 mm field-of-view (FOV) (160 spatial locations) to record both a RLPSW and a surface wave in the first 50 ms after the excitation initiation. To achieve a two-dimensional (2D) elastography measurement of scaffolds in a fast manner for tissue engineering, the lateral spacing was chosen to cover a 2-4 mm FOV (20-40 spatial locations) for each spatial position so that AFEM can take data over a small patch centered about the excitation area for each spatial position to achieve a high throughput measurement with AFEM. The scan pattern of AFEM (FIG. 1C) at each individual location, p, a data set was acquired with dimensions of z depth samples and N=i·j is the product of i M-scans and/B-scans (M-B scan) for an elasticity measurement in each location. For 2D scans, the scan pattern was composed of a dataset with the dimensions of z by N by k by s matrix, where k and s are the number of lateral and elevational elastography measurement positions, respectively. The 2D AFEM measurement can reveal more details of the elastic properties of scaffolds in tissue engineering, especially for composite scaffolds due to heterogeneous properties.

As described above and illustrated again in FIG. 2, in an AFEM system the LPSWs are generated by the acoustic radiation force, and the RLPSW traveling through the sample thickness h is detected at the surface by OCT, ultrasound, or other motion detection techniques. The wave velocity traveling in the thin layer solid materials can be evaluated using:

Velocity=Depth/(0.5tₐ)

where tₐ is the arrival time of the reflected wave measured at the surface of the material. This process can be repeated at any point within a medium to obtain a point-by-point measurement.

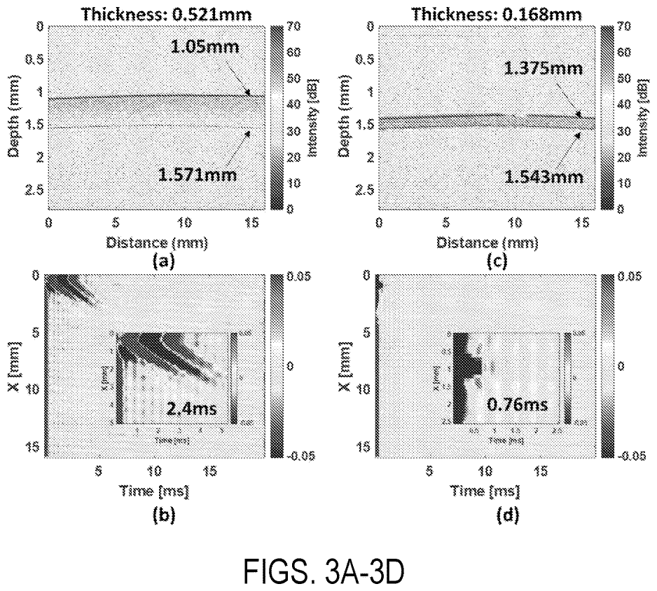

AFEM is able to measure samples with various small thicknesses. In an example study, a 5% gelatin tissue-mimicking thin samples with two thickness, 520 μm (FIG. 3A) and 170 μm thickness (FIG. 3C), were fabricated to demonstrate the performance of AFEM. The reflected waves were recorded by OCT, presented in FIG. 3B for 520 μm thickness and in FIG. 3D for 170 μm thickness. The arrival time of the reflected waves was 2.4 ms for 520 μm thickness and 0.76 ms for 170 μm thickness. The velocities that were measured in the samples with 520 μm and 170 μm are 0.43 m/s and 0.44 m/s, respectively.

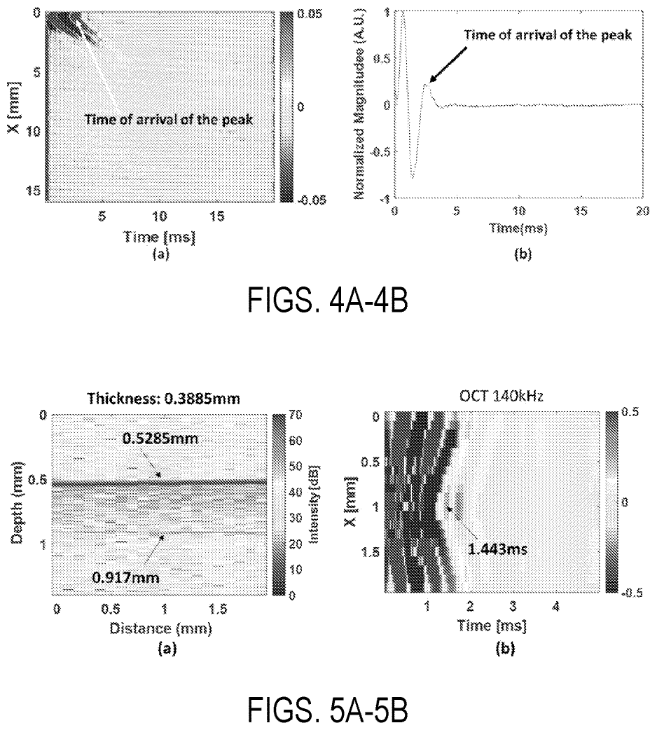

A profile at x=1 mm is illustrated to demonstrate the peak for arrival time of reflected waves traveling through the depth and coming back to the surface (FIGS. 4A and 4B) as an important feature. The thickness of the sample can be determined from the OCT image (FIG. 5A). The measured data produced by AFEM shown in FIG. 5B (only 44 MB) is much smaller than regular optical coherence elastography data set (2.5 GB) using a 16 mm wide field-of-view with a 50 ms recording period. The imaging range using AFEM is 2 mm wide and 5 ms recording period. This could be further reduced with optimization of alignment of the ultrasound transducer and OCT (or other motion detection) system such that only one location (or a very limited number of locations) would need to be used for the measurement.

The AFEM systems and methods described in the present disclosure can acquire both 4D (x, y, z, t) data and evaluate localized elasticity on a point-by-point basis of thin layer materials to obtain a high resolution elastography map compared with regular optical coherence elastography or other elastography techniques. In an example implementation, the AFEM scan rate may be 140 kHz with a 2 mm scan range for each point. The lateral spacing (x) of the elastography map in these instances may be 0.635 mm and the elevation spacing (y) may be 1.27 mm. The scan range in these instances may be 13.97 mm×13.97 mm and the scanning may be performed using manual translation of the sample, or automatically with a motorized translation stage.

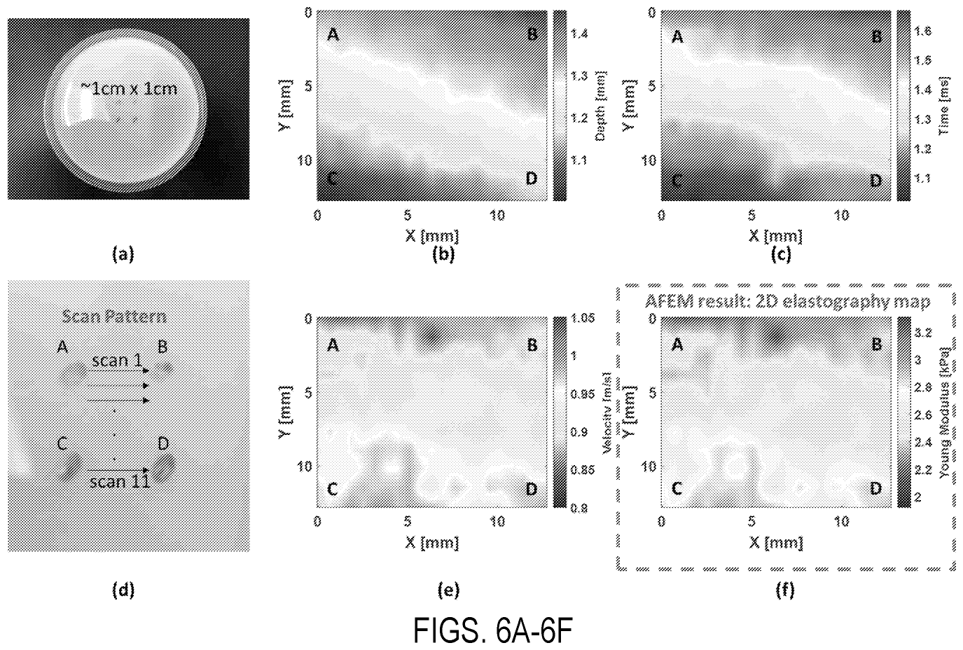

FIG. 6A shows a 5% gelatin phantom with approximately 1.2 mm thickness, and the scan range was marked by red dots (FIG. 6D). The 2D map of the phantom thickness or depth is shown in FIG. 6B. The 2D map of the arrival time, tₐ, is presented in FIG. 6C. Using the depth and arrival time information, the wave velocity can be calculated and is shown in FIG. 6E. Eventually, the localized 2D elastography map (FIG. 6F), Young's modulus, can be determined using the relationship E=3 ρV², where Vis the wave velocity. This assumes that the material is isotropic, homogeneous, and nearly incompressible.

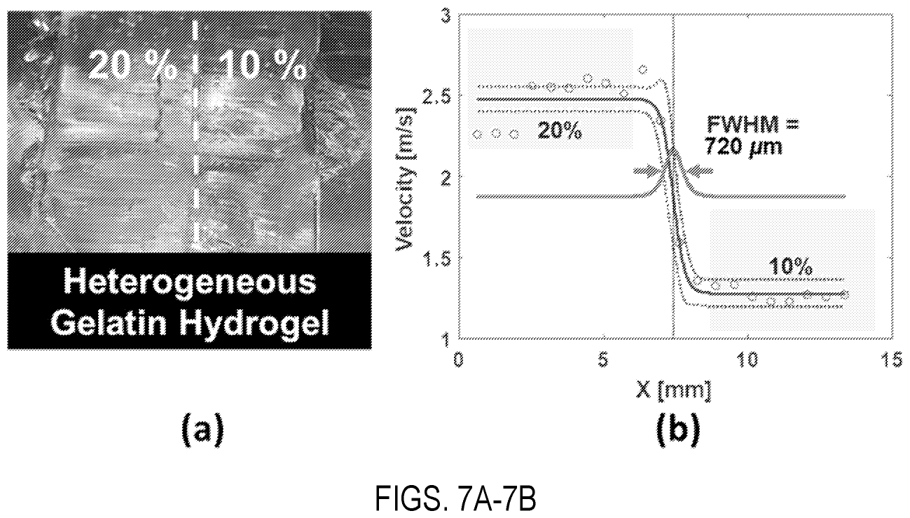

FIGS. 7A-7B show an example evaluation of the elastography resolution of AFEM in a gelatin hydrogel with high mechanical contrast. A heterogenous gelatin hydrogel sample with high elastic contrast (10% v/v versus 20% v/v) was fabricated using gelatin powder (gel strength 300 type A, G2500-1 KG, Sigma-Aldrich, St. Louis, MO, USA). Due to advantage of the AFEM, the scatterers are not needed, and the hydrogel can be completely transparent. For 20% gelatin hydrogel, a total volume of 100 mL tap water in a 500 mL beaker was heated to approximate 70 °C and the gelatin powders was added with stirring to the beaker for approximately five minutes to homogenize the solution. The gelatin solution was placed in a de-gassing chamber to remove small bubbles in the fluid and then was poured into a container (15 mm diameter×10 mm height). The liquid gelatin hydrogel was transferred to a 4 °C refrigerator for 3 hours for congealing. After 3 hours, the above procedures were repeated for 10% gelatin hydrogel. The liquid gelatin hydrogel was poured into the same container to cover the substrate (20% gelatin hydrogel), which was transferred to a 4 °C refrigerator for the other 3 hours for congealing. Two planes with approximately 2 mm distance were sliced by using the tissue slicer blade to make a thin gelatin hydrogel sample and boundary was indicated with white dash line, presented in FIG. 7A. A total of 21 measurements with the step of 0.635 mm were performed and the 11th measurement was exactly at the boundary. The scan rate of the AFEM was 50 kHz with 50 μm lateral resolution. The acoustic excitation was set as 50 μs to achieve a sharp temporal resolution. The velocity profile was fit to a sigmoid function as an edge-spread function (FIG. 7B) for the calculation of the elastography spatial resolution of the AFEM based on full-width half-maximum (FWHM) of the differentiated edge-spread function and the elastography spatial resolution was approximately evaluated as 720 μm.

In addition to an OCT system or optical methods used for detection of the wave motion, other methods could be used for obtaining data for evaluating the wave propagation including using ultrasound with a single element transducer or an array transducer. Pulse-echo imaging techniques can be used to obtain data necessary for evaluating incremental motion steps.

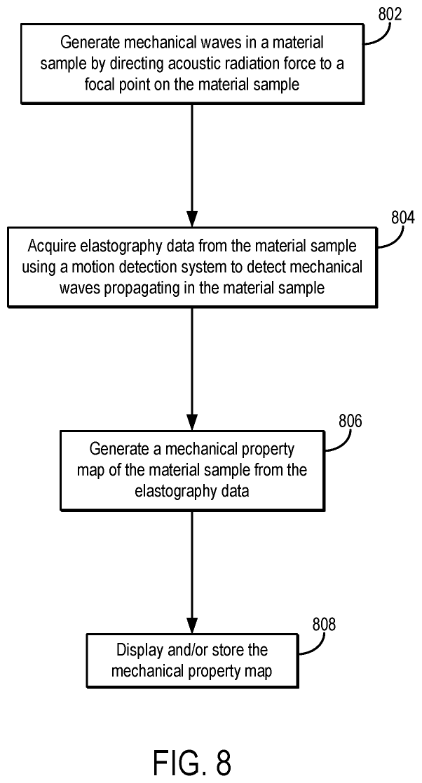

Referring now to FIG. 8, a flowchart is illustrated as setting forth the steps of an example method for acoustic force elastography microscopy.

The method includes generating an acoustic force with an ultrasound transducer and directing the acoustic force onto a material sample, as indicated at step 802. As a result, mechanical waves are generated and begin propagating within the material sample. The mechanical waves may include surface waves propagating on a surface of the material sample, as well as LPSWs and RLPSWs propagating along a thickness of the material sample.

While the mechanical waves are propagating within the material sample, elastography data are acquired using a suitable motion detection system, as indicated at step 804. As one non-limiting example, the motion detection system is an OCT system. In these instances, the elastography data may be acquired by measuring the motion (e.g., displacement, velocity, and/or acceleration) in the material sample caused by the propagating mechanical waves using light generated by a light source of the OCT system.

As another non-limiting example, the motion detection system may be an ultrasound system. The ultrasound system may be the same ultrasound system used to generate the acoustic force, or may be a separate ultrasound system. In these instances, the elastography data may be acquired by measuring the motion (e.g., displacement, velocity, and/or acceleration) in the material sample caused by the propagating mechanical waves using ultrasound waves transmitted into the material sample. The ultrasound waves may be focused ultrasound waves, unfocused ultrasound waves, or combinations thereof. When the same ultrasound system is used to both generate the acoustic force and measure the elastography data, the ultrasound system may be switched between an acoustic force mode and an elastography mode, or in some instances may be operated in a parallel mode when the acoustic force is generated while elastography data are also being acquired (e.g., by using different transducers of the same ultrasound system, using different elements of a single ultrasound transducer, etc.).

As described above, the elastography data may include measurements of displacement within the material sample, measurements of mechanical wave velocity, and so on. As one example, the elastography data may include a measurement of depth in the material sample and a corresponding measurement of arrival time, tₐ, for the RLPSWs generated in the material sample. From these depth and arrival time measurements, the mechanical wave velocity can be computed, as described above. The wave velocity may then be stored as part of the elastography data.

The elastography data are then processed with the computer system to generate a mechanical property map for the material sample, as indicated at step 806. As described above, in one example the mechanical property map may include an elasticity or Young's modulus map whose pixels indicate the spatial distribution of elasticity or Young's modulus values within the material sample. The mechanical properties depicted in the mechanical property map may be calculated on a pixel-by-pixel basis.

The mechanical property map may then be displayed to a user, or stored by the computer system for later use and/or further processing, as indicated at step 808.

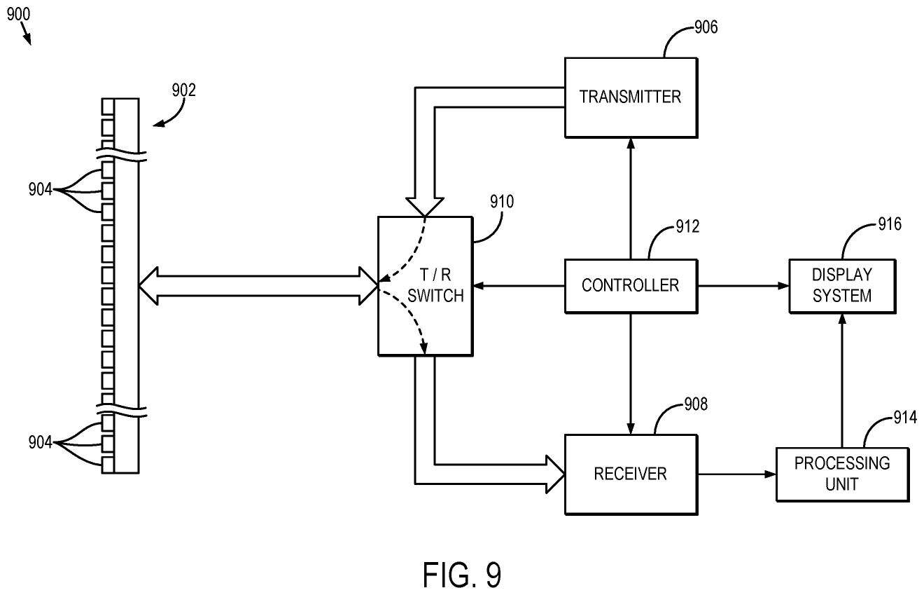

FIG. 9 illustrates an example of an ultrasound system 900 that can be used when implementing the methods described in the present disclosure. The ultrasound system 900 may be used to generate the ARF 104 for generating mechanical waves in a material sample 106, may be used as the motion detection system 130 for detecting or otherwise measuring those mechanical waves, or both.

In general, the ultrasound system 900 includes a transducer array 902 that includes a plurality of separately driven transducer elements 904. The transducer array 902 can include any suitable ultrasound transducer array, including linear arrays, curved arrays, phased arrays, and so on. Similarly, the transducer array 902 can include a 1D transducer, a 1.5D transducer, a 1.75D transducer, a 2D transducer, a 3D transducer, and so on. In some other implementations, the transducer array 902 may instead include an ultrasound transducer with a single transducer element 904.

When energized by a transmitter 906, a given transducer element 904 produces a burst of ultrasonic energy. The ultrasonic energy reflected back to the transducer array 902 (e.g., an echo) from the object or subject under study may be converted to an electrical signal (e.g., an echo signal) by each transducer element 904 and can be applied separately to a receiver 908 through a set of switches 910. The transmitter 906, receiver 908, and switches 910 are operated under the control of a controller 912, which may include one or more processors. As one example, the controller 912 can include a computer system.

The transmitter 906 can be programmed to transmit unfocused or focused ultrasound waves. In some configurations, the transmitter 906 can also be programmed to transmit diverged waves, spherical waves, cylindrical waves, plane waves, or combinations thereof. Furthermore, the transmitter 906 can be programmed to transmit spatially or temporally encoded pulses.

The receiver 908 can be programmed to implement a suitable detection sequence for the imaging task at hand. In some embodiments, the detection sequence can include one or more of line-by-line scanning, compounding plane wave imaging, synthetic aperture imaging, and compounding diverging beam imaging.

In some configurations, the transmitter 906 and the receiver 908 can be programmed to implement a high frame rate. For instance, a frame rate associated with an acquisition pulse repetition frequency (“PRF”) of at least 100 Hz can be implemented. In some configurations, the ultrasound system 900 can sample and store at least one hundred ensembles of echo signals in the temporal direction.

A scan can be performed by setting the switches 910 to their transmit position, thereby directing the transmitter 906 to be turned on momentarily to energize transducer elements 904 during a single transmission event according to a selecting imaging sequence. The switches 910 can then be set to their receive position and the subsequent echo signals produced by the transducer elements 904 in response to one or more detected echoes are measured and applied to the receiver 908. The separate echo signals from the transducer elements 904 can be combined in the receiver 908 to produce a single echo signal.

The echo signals are communicated to a processing unit 914, which may be implemented by a hardware processor and memory, to process echo signals or images generated from echo signals. As an example, the processing unit 914 can receive ultrasound signals measured by the ultrasound transducer 902 as elastography data indicative of mechanical wave motion in the material sample 106. These elastography data can be processed by the processing unit 914 to generate elasticity maps or other mechanical property maps or measurements using the methods described in the present disclosure. Images, maps, and/or measurement data produced by the processing unit 914 can be displayed on a display system 916.

As described above, in some implementations the ultrasound system 900 may be used to generate an acoustic radiation force for inducing mechanical waves in the material sample. In these instances, the ultrasound transducer 902 may include a single transducer element 904 for generating a focused ultrasound beam, or may include a plurality of transducer elements 904 energized to generate a focused ultrasound beam. Additionally or alternatively, the ultrasound system 900 may be used to detect motion in the material sample caused by the propagating mechanical waves. For instance, the ultrasound system 900 may be used to measure the depth of the material sample, the time of arrival of RLPSWs, and so on.

The present disclosure has described one or more preferred embodiments, and it should be appreciated that many equivalents, alternatives, variations, and modifications, aside from those expressly stated, are possible and within the scope of the invention.

1. An acoustic force elastography microscope system, comprising:
a translation stage to hold a material sample and to translate the material sample along a first spatial dimension and a second spatial dimension orthogonal to the first spatial dimension;
an ultrasound transducer to generate an acoustic radiation force (ARF) directed at a surface of the material sample; and
an optical coherence tomography system to acquire elastography data by measuring particle motion attributable to shear wave motion in the material sample caused by the ARF.

2. The acoustic force elastography microscope of claim 1, wherein the ultrasound transducer comprises a single transducer element.

3. The acoustic force elastography microscope of claim 1, wherein the ultrasound transducer generates the ARF using a focused ultrasound beam directed at a focal point on the surface of the material sample.

4. The acoustic force elastography microscope of claim 1, comprising a computer system that receives the elastography data from the optical coherence tomography system and generates an elasticity map therefrom.

5. The acoustic force elastography microscope of claim 4, wherein the computer system generates the elasticity map by computing shear wave velocity values from the elastography data and generating the elasticity map from the shear wave velocity values.

6. An acoustic force elastography microscope system, comprising:
a translation stage configured to hold a material sample and to translate the material sample along a first spatial dimension and a second spatial dimension orthogonal to the first spatial dimension;
an ultrasound system to:
generate an acoustic radiation force (ARF) directed at a surface of the material sample; and
acquire elastography data by measuring particle motion attributable to shear wave motion in the material sample caused by the ARF.

7. The acoustic force elastography microscope of claim 6, wherein the ultrasound system comprises a first ultrasound transducer to generate the ARF and a second ultrasound transducer to acquire the elastography data.

8. The acoustic force elastography microscope of claim 7, wherein the first ultrasound transducer comprises a single transducer element.

9. The acoustic force elastography microscope of claim 7, wherein the first ultrasound transducer generates the ARF using a focused ultrasound beam directed at a focal point on the surface of the material sample.

10. The acoustic force elastography microscope of claim 6, comprising a computer system that receives the elastography data from the ultrasound system and generates an elasticity map therefrom.

11. The acoustic force elastography microscope of claim 10, wherein the computer system generates the elasticity map by computing shear wave velocity values from the elastography data and generating the elasticity map from the shear wave velocity values.

12. A method for acoustic force elastography microscopy, the method comprising:
(a) generating an acoustic radiation force (ARF) with an ultrasound transducer, the ARF being exerted on a surface of a material thereby generating surface waves that travel outwards on the surface and a longitudinally polarized shear wave that travels through a thickness of the material;
(b) acquiring elastography data to measure particle motion of reflected longitudinally polarized shear waves generated by the ARF; and
(c) reconstructing a mechanical property map from the elastography data.

13. The method of claim 12, wherein the elastography data are acquired using an optical coherence tomography system.

14. The method of claim 12, wherein the elastography data are acquired using an ultrasound system.

15. The method of claim 12, wherein the elastography data comprise depth measurements indicating depth in the material and arrival time measurements indicating arrival times of reflected longitudinally polarized shear waves generated by the ARF.

16. The method of claim 15, wherein reconstructing the mechanical property map comprises generating shear wave velocity measurement data from the depth measurements and the arrival time measurements, wherein the mechanical property map is reconstructed from the shear wave velocity measurement data.

17. The method of claim 12, wherein the mechanical property map comprises an elasticity map depicting measurements of elasticity at spatial locations in the material.

18. The method of claim 12, wherein the material comprises a hydrogel.

19. The method of claim 18, wherein the hydrogel is an optically transparent hydrogel.

20. The method of claim 19, wherein the elastography data are acquired using an optical coherence tomography system.

21. The method of claim 18, wherein the hydrogel comprises at least one of a hydrogel scaffold, a tissue-mimicking gelatin hydrogel, an oligo[poly(ethylene glycol)fumarate] (OPF) hydrogel, or an OPF hydroxyapatite (HA) nanocomposite.

22. The method of claim 21, wherein the hydrogel comprises a cell-laden hydrogel scaffold.

23. The method of claim 12, wherein steps (a)-(c) are repeated at different times using the same material to generate a plurality of mechanical property maps of the material.
